(19) United States
(12) Patent Application Publication
(10) Pub. No.: US 2025/0195593 A1
(43) Pub. Date: Jun. 19, 2025

(54) METHOD FOR INHIBITING GROWTH OF CANCER CELLS USING SUPERCRITICAL FLUID-EXTRACTED PRODUCT OF AGARICUS BLAZEI MURRILL

(71) Applicants: Chi-Ming KAO, Taipei City (TW); I-MEI FOODS CO., LTD., Taipei City (TW)

(72) Inventor: Ya-Chi TSAI, Taipei City (TW)

(21) Appl. No.: 18/603,435

(22) Filed: Mar. 13, 2024

(30) Foreign Application Priority Data
Dec. 18, 2023 (TW) 112149268

(51) Int. Cl. A61K 36/07; A61K 36/074; A61K 36/9066; A61P 35/00

(52) U.S. Cl. CPC A61K 36/07; A61K 36/074; A61K 36/9066; A61P 35/00; A61K 2236/33; A61K 2236/37

(57) ABSTRACT

A method for inhibiting the growth of cancer cells includes administering to a subject in need thereof a pharmaceutical composition containing a supercritical fluid-extracted product of Agaricus blazei Murrill. The pharmaceutical composition further contains an ethanol-extracted product of Curcuma longa and a supercritical fluid-extracted product of Ganoderma lucidum. The pharmaceutical composition is in a dosage form selected from the group consisting of a parenteral dosage form, an oral dosage form, and a topical dosage form.

### CROSS-REFERENCE TO RELATED APPLICATION

This application claims priority to Taiwanese Invention patent application No. 112149268, filed on Dec. 18, 2023, which is incorporated by reference herein in its entirety.

### FIELD

The present disclosure relates to a method for inhibiting the growth of cancer cells using a supercritical fluid-extracted product of Agaricus blazei Murrill.

### BACKGROUND

Nowadays, cancer is the leading cause of death worldwide. Although the exact formation mechanism of cancer remains not fully understood, it is acknowledged that carcinogenesis or tumorigenesis can be attributed to genetic mutation caused by the accumulation of exogenous or endogenous factors within cells. When genetic mutation occurs, signal transduction pathways within the cells may be disrupted, causing cell division to go out of control, resulting in abnormal cell proliferation and gradual formation of cancer cells.

Although there are numerous drugs currently in clinical use to treat cancer, such as cisplatin, doxorubicin, and paclitaxel, the cure rates for cancer remain limited. The primary reasons behind this limitation include individual variations among patients, severe side effects associated with anti-cancer drugs, and drug resistance of the cancer cells.

Agaricus blazei Murrill, also known as Agaricus subrufescens, Agaricus brasiliensis, and Agaricus rufotegulis, is a member of the genus Agaricus in family Agaricaceae, and is an edible and medicinal fungi mainly distributed in Brazil, Peru, and Southern California in the United States. Previous studies have demonstrated that Agaricus blazei Murrill can exhibit a wide range of biological activities, such as antioxidant, anti-inflammatory, hypoglycemic, and hypolipidemic activities.

It has been reported in Kim C. F. et al. (2009), J. Ethnopharmacol., 122:320-326 that an ethanol/water-extracted product of Agaricus blazei Murrill could inhibit the growth of cancer cells in nude mice bearing xenogenic human promyelocytic leukemia NB-4 cells. In addition, it has been reported in Mazzutti S. et al. (2012), J. Supercrit. Fluids, 70:48-56 that a supercritical fluid-extracted product of Agaricus blazei Murrill had antimicrobial activity.

In spite of the aforesaid, there is still a need to develop an effective way for inhibiting the growth of cancer cells.

### SUMMARY

Therefore, an object of the present disclosure is to provide a method for inhibiting the growth of cancer cells, which can alleviate at least one of the drawbacks of the prior art, and which includes administering to a subject in need thereof a pharmaceutical composition containing a supercritical fluid-extracted product of Agaricus blazei Murrill.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the high performance liquid chromatography (HPLC) spectrum of the supercritical fluid-extracted product of Agaricus blazei Murrill.

FIG. 2 shows the cell viability percentages of the A549 cells, AGS cells, SW480 cells, or MDA-MB-231 cells determined in each group of Example 3, infra.

FIG. 3 shows the cell viability percentages of the MIA Paca-2 cells determined in each group of Example 4, infra, in which the symbol “***” represents p<0.001 (compared with the control group 1), the symbol “###” represents p<0.001 (compared with the experimental group 1-1), the symbol “+++” represents p<0.001 (compared with the experimental group 2-1), the symbol “$” represents p<0.05 (compared between the experimental groups 1-1 and 2-1), and the symbol “$$$” represents p<0.001 (compared between the experimental groups 3-1 and 4-1).

FIG. 4 shows the cell viability percentages of the HeLa 229 cells determined in each group of Example 4, infra, in which the symbol “**” represents p<0.01 (compared with the control group 2), the symbol “***” represents p<0.001 (compared with the control group 2), the symbol “#” represents p<0.05 (compared with the experimental group 1-2), the symbol “+++” represents p<0.001 (compared with the experimental group 2-2), and the symbol “$$$” represents p<0.001 (compared between the experimental groups 3-2 and 4-2).

FIG. 5 shows the cell viability percentages of the PC-3 cells determined in each group of Example 4, infra, in which the symbol “*” represents p<0.05 (compared with the control group 3), the symbol “**” represents p<0.01 (compared with the control group 3), the symbol “***” represents p<0.001 (compared with the control group 3), the symbol “#” represents p<0.05 (compared with the experimental group 1-3), the symbol “+++” represents p<0.001 (compared with the experimental group 2-3), the symbol “$” represents p<0.05 (compared between the experimental groups 1-3 and 2-3), and the symbol “$$$” represents p<0.001 (compared between the experimental groups 3-3 and 4-3).

FIG. 6 shows the cell viability percentages of the A549 cells, AGS cells, HepG2 cells, SW480 cells, and MDA-MB-231 cells determined in each group of Example 4, infra.

FIG. 7 shows the changes in A549 tumor volume over time determined in each group of nude mice of Example 5, infra, in which the symbol “*” represents p<0.05 (compared with the control group), the symbol “**” represents p<0.01 (compared with the control group), the symbol “***” represents p<0.001 (compared with the control group), the symbol “#” represents p<0.05 (compared with the experimental group 1), and the symbol “##” represents p<0.01 (compared 15 with the experimental group 1).

FIG. 8 shows the changes in MIA Paca-2 tumor volume over time determined in each group of nude mice of Example 5, infra, in which the symbol represents p<0.05 (compared with the control group), the symbol “**” represents p<0.01 (compared with the control group), and the symbol “***” represents p<0.001 (compared with the control group).

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for inhibiting the growth of cancer cells, which includes administering to a subject in need thereof a pharmaceutical composition containing a supercritical fluid-extracted product of Agaricus blazei Murrill.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats.

According to the present disclosure, the cancer cells may be selected from the group consisting of lung adenocarcinoma cells, gastric adenocarcinoma cells, colorectal cancer cells, breast cancer cells, liver cancer cells, pancreatic cancer cells, prostate cancer cells, cervical cancer cells, and combinations thereof.

According to the present disclosure, the pharmaceutical composition may further contain an extracted product selected from the group consisting of an ethanol-extracted product of Curcuma longa, a supercritical fluid-extracted product of Ganoderma lucidum, and a combination thereof.

In certain embodiments, the pharmaceutical composition contains the supercritical fluid-extracted product of Agaricus blazei Murrill and the ethanol-extracted product of Curcuma longa. In certain embodiments, the supercritical fluid-extracted product of Agaricus blazei Murrill and the ethanol-extracted product of Curcuma longa in the pharmaceutical composition are present in a weight ratio ranging from 1:1 to 1:5. In an exemplary embodiment, the weight ratio of the supercritical fluid-extracted product of Agaricus blazei Murrill and the ethanol-extracted product of Curcuma longa is 1:1.

In certain embodiments, the pharmaceutical composition contains the supercritical fluid-extracted product of Agaricus blazei Murrill and the supercritical fluid-extracted product of Ganoderma lucidum. In certain embodiments, the supercritical fluid-extracted product of Agaricus blazei Murrill and the supercritical fluid-extracted product of Ganoderma lucidum in the pharmaceutical composition are present in a weight ratio ranging from 1:1 to 1:5. In an exemplary embodiment, the weight ratio of the supercritical fluid-extracted product of Agaricus blazei Murrill and the supercritical fluid-extracted product of Ganoderma lucidum longa is 1:1.

In certain embodiments, the pharmaceutical composition contains the supercritical fluid-extracted product of Agaricus blazei Murrill, the ethanol-extracted product of Curcuma longa, and the supercritical fluid-extracted product of Ganoderma lucidum. In certain embodiments, the supercritical fluid-extracted product of Agaricus blazei Murrill, the ethanol-extracted product of Curcuma longa, and the supercritical fluid-extracted product of Ganoderma lucidum in the pharmaceutical composition are present in a weight ratio ranging from 1:1:1 to 1:5:1. In an exemplary embodiment, the weight ratio of the supercritical fluid-extracted product of Agaricus blazei Murrill, the ethanol-extracted product of Curcuma longa, and the supercritical fluid-extracted product of Ganoderma lucidum is 1:1:1. In another exemplary embodiment, the weight ratio of the supercritical fluid-extracted product of Agaricus blazei Murrill, the ethanol-extracted product of Curcuma longa, and the supercritical fluid-extracted product of Ganoderma lucidum is 1:2:1.

According to the present disclosure, the supercritical fluid extraction (SFE) method for obtaining the supercritical fluid-extracted product of Agaricus blazei Murrill and the supercritical fluid-extracted product of Ganoderma lucidum is within the expertise and routine skills of those skills in the art (for example, see Mazzutti S. et al. (2012), J. Supercrit. Fluids, 70:48-56). It should be noted that for achieving the best extraction results, the procedures and operating conditions for the SFE method may be adjusted according to practical requirements and relevant factors (such as the type of the supercritical fluid, and the amount and the type of the fruiting bodies of Agaricus blazei Murrill and Ganoderma lucidum).

According to the present disclosure, the supercritical fluid may be selected from the group consisting of supercritical carbon dioxide (CO₂), water (H₂O), methanol, and ethanol. In certain embodiments, the supercritical fluid is supercritical CO₂.

According to the present disclosure, Agaricus blazei Murrill and Ganoderma lucidum may be independently a fresh, unprocessed fruiting body, or may be prepared by a process selected from the group consisting of a drying treatment (such as a freeze-drying treatment), a heating treatment, a grinding treatment, a chopping treatment, a comminuting treatment, and combinations thereof.

In certain embodiments, the supercritical fluid-extracted product of Agaricus blazei Murrill may be obtained by subjecting freeze-dried fruiting bodies of Agaricus blazei Murrill to a supercritical fluid extraction treatment.

In certain embodiments, the supercritical fluid-extracted product of Ganoderma lucidum may be obtained by subjecting freeze-dried fruiting bodies of Ganoderma lucidum to a supercritical fluid extraction treatment.

According to the present disclosure, the supercritical fluid extraction treatment may be conducted at a temperature ranging from 40° C. to 60° C. and a pressure ranging from 1071.33 psi to 4350 psi for 30 minutes to 960 minutes. In an exemplary embodiment, the supercritical fluid extraction treatment is conducted at a temperature of 60° C. and a pressure of 4350 psi for 120 minutes.

According to the present disclosure, the ethanol extraction method for obtaining the ethanol-extracted product of Curcuma longa is within the expertise and routine skills of those skills in the art (for example, see TW 1748565 B). It should be noted that for achieving the best extraction results, the procedures and operating conditions for the ethanol extraction method may be adjusted according to practical requirements and relevant factors (such as the type and the amount of the plant material, and the amount of the ethanol).

According to the present disclosure, Curcuma longa may be a fresh, unprocessed material (such as the rhizome, leaf, flower, or whole plant of Curcuma longa), or may be prepared by a process selected from the group consisting of a drying treatment (such as a freeze-drying treatment), a heating treatment, a grinding treatment, a chopping treatment, a comminuting treatment, and combinations thereof.

In certain embodiments, the ethanol-extracted product of Curcuma longa may be obtained by subjecting freeze-dried rhizome of Curcuma longa to an ethanol extraction treatment.

According to the present disclosure, the ethanol extraction treatment may be conducted at a temperature ranging from 10° C. to 100° C. for a time period ranging from 15 minutes to 150 minutes. In an exemplary embodiment, the ethanol extraction treatment is conducted at 25° C. for 30 minutes.

According to the present disclosure, the pharmaceutical composition may be formulated into a dosage form suitable for oral administration, parenteral administration, or topical administration using technology well known to those skilled in the art.

According to the present disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intrapleural injection, intramuscular injection, intravenous injection, intraarterial injection, intraarticular injection, intrasynovial injection, intrathecal injection, intracranial injection, intraepidermal injection, subcutaneous injection, intradermal injection, intralesional injection, and sublingual administration.

According to the present disclosure, the pharmaceutical composition may be formulated into an external preparation suitable for topical application to the skin using technology well known to those skilled in the art. The external preparation includes, but is not limited to, emulsions, gels, ointments, creams, patches, liniments, powder, aerosols, sprays, lotions, serums, pastes, foams, drops, suspensions, salves, and bandages.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

The dose and frequency of administration of the pharmaceutical composition of the present disclosure may vary depending on the following factors: the severity of the illness or disorder to be treated, routes of administration, and age, physical condition and response of the subject to be treated. In general, the pharmaceutical composition may be administered in a single dose or in several doses.

The disclosure will be further described by way of the following examples. However, it should be understood that the following examples are solely intended for the purpose of illustration and should not be construed as limiting the disclosure in practice.

### EXAMPLES

### General Experimental Materials

### 1. Source and Cultivation of Cell Lines

Eight cell lines used in the following experiments were purchased from the Bioresource Collection and Research Center (BCRC) of the Food Industry Research and Development Institute (FIRDI) (No. 331, Shih-Pin Rd., Hsinchu City 300, Taiwan). The relevant information regarding each of the cell lines is listed in Table 1 below.

TABLE 1

| Cell line | Accession number |
|---|---|
| Human pancreatic cancer cell line MIA Paca-2 | BCRC 60139 |
| Human prostate cancer cell line PC-3 | BCRC 60122 |
| Human cervical cancer cell line HeLa 229 | BCRC 60290 |
| Human lung adenocarcinoma cell line A549 | BCRC 60074, |
| | corresponding to |
| | ATCC CCL-185 |
| Human hepatocellular carcinoma cell line HepG2 | BCRC RM60025 |
| Human colorectal cancer cell line SW480 | BCRC 60249 |
| Human breast cancer cell line MDA-MB-231 | BCRC 60425 |
| Human gastric adenocarcinoma cell line AGS | BCRC 60102 |

A respective one of the eight cell lines was cultivated using the corresponding medium as shown in Table 2 in an incubator with culture conditions set at 37° C. and 5% CO₂. Medium change was performed every two to three days. Cell passage was performed when the cultured cells reached 80% of confluence.

TABLE 2

| Cell line | Medium | |
|---|---|---|
| MIA Paca-2 | Dulbecco's Modified Eagle's Medium (DMEM, | |
| cells | Hyclone) supplemented with 10% fetal bovine serum | |
| | (FBS, Gibco), 2.5% horse serum (HS, Gibco) and 1% | |
| | antibiotic-antimycotic solution (Hyclone) | |
| PC-3 cells | Ham's F-12K nutrient mixture medium (Kaighn's | |
| | modification of Ham's F-12 nutrient mixture medium, | |
| | Hyclone) supplemented with 7% FBS (Gibco) and 1% | |
| | antibiotic-antimycotic solution (Hyclone) | |
| HeLa 229 | Minimum Essential Medium/Eagle's balanced salt | |
| cells | solution with nonessential amino acid (MEM/EBSS | |
| | with NEAA, Hyclone) supplemented with 10% FBS | |
| | (Gibco) | |
| A549 cells | For | DMEM (Hyclone) supplemented with 5% |
| | Example 3 | FBS (Gibco) |
| | and | |
| | Example 4 | |
| | For | Ham's F-12K nutrient mixture medium |
| | Example 5 | (Kaighn's modification of Ham's F-12 |
| | | nutrient mixture medium, Hyclone) |
| | | supplemented with 10% FBS (Gibco) |
| | | and 1% antibiotic-antimycotic solution |
| | | (Hyclone) |
| HepG2 cells | DMEM (Hyclone) supplemented with 5% FBS (Gibco) | |
| SW480 cells | | |
| MDA-MB-231 | | |
| cells | | |
| AGS cells | | |

### General Procedures

### 1. Statistical Analysis

All the experiments described below were performed in triplicates. The experimental data of all the test groups are expressed as mean±standard error of the mean (SEM), and were analyzed using Student's t-test, so as to evaluate the differences between the groups. Statistical significance is indicated by p<0.05.

### Example 1. Preparation of Supercritical Fluid-Extracted Product of Agaricus blazei Murrill

First, fruiting bodies of Agaricus blazei Murrill purchased from Lohas Biotech Development Corp. were washed with deionized water, and then subjected to a freeze-drying treatment for 48 hours. The resultant freeze-dried fruiting bodies of Agaricus blazei Murrill were subsequently subjected to a grinding treatment using a grinding machine (Manufacturer: Tinso, Model no.: TS10HS), followed by filtering through a sieve with a porosity of 0.38 mm, so as to obtain a freeze-dried powder of Agaricus blazei Murrill.

Next, approximately 9 kg of the freeze-dried powder of Agaricus blazei Murrill was placed in an extraction vessel of a supercritical fluid extraction system (Manufacturer: Taiwan Supercritical Technologies Co., Ltd., Model no.: SE-201C), followed by subjecting the freeze-dried powder of Agaricus blazei Murrill to a supercritical fluid extraction treatment using supercritical carbon dioxide (CO₂) at 60° C. and 4350 psi for 120 minutes. During the supercritical fluid extraction treatment, the supercritical CO₂ was introduced to pass through the extraction vessel at a flow rate of 2.5 L/min, and then entered a collection vessel of the supercritical fluid extraction system at atmospheric pressure, so that a supercritical fluid-extracted product of Agaricus blazei Murrill in pasty form was obtained in the collection vessel.

### Example 2. High Performance Liquid Chromatography (HPLC) Analysis

### Experimental Procedures

The supercritical fluid-extracted product of Agaricus blazei Murrill in pasty form obtained in Example 1 was dissolved in an appropriate amount of isopropyl alcohol (IPA), thereby obtaining a test sample of the supercritical fluid-extracted product of Agaricus blazei Murrill having a concentration of 0.2 g/mL.

The test sample was subjected to high performance liquid chromatography (HPLC) analysis using technology well-known to those skilled in the art. The operating parameters and conditions for performing HPLC are summarized in Table 3 below.

TABLE 3

| | |
|---|---|
| HPLC instrument | HPLC system (Manufacturer: Thermo Fisher Scientific, |
| | Model no.: Ultimate 3000) equipped with a pump |
| | (Manufacturer: Thermo Fisher Scientific, Model no.: |
| | HPG-3200BX) and a Rapid Separation (RS) detector |
| | (Manufacturer: Thermo Fisher Scientific, Model no.: |
| | VWD-3400RS) |
| Type of | Hypersil GOLD aQ Column (Manufacturer: Thermo |
| chromatography | Fisher Scientific, Part no.: 25305-159270A) |
| column | |
| Size of | Length: 150 mm; |
| chromatography | inner diameter: 20 mm |
| column | |
| Detection | 230 nm (ultraviolet light) |
| wavelength | |
| Mobile phase | Ethanol (A)/ultrapure water (B) (10:90, v/v) |
| Gradient | The mobile phase was conducted for 50 minutes as |
| elution | follows: A:B was 10:90 (v/v) during the 0^{th} minute to 1^{st} |
| | minute, A:B changed from 10:90 (v/v) to 99:1 (v/v) |
| | during the 1^{st} minute to 12^{th} minute, A:B was 99:1 (v/v) |
| | during the 12^{th} minute to 42^{nd} minute, A:B changed |
| | from 99:1 (v/v) to 10:90 (v/v) during the 42^{nd} minute to |
| | 45^{th} minute, and A:B was 10:90 (v/v) during the 45^{th} |
| | minute to 50^{th} minute. |
| Flow rate of | 10 mL/min |
| test sample | |

### Results

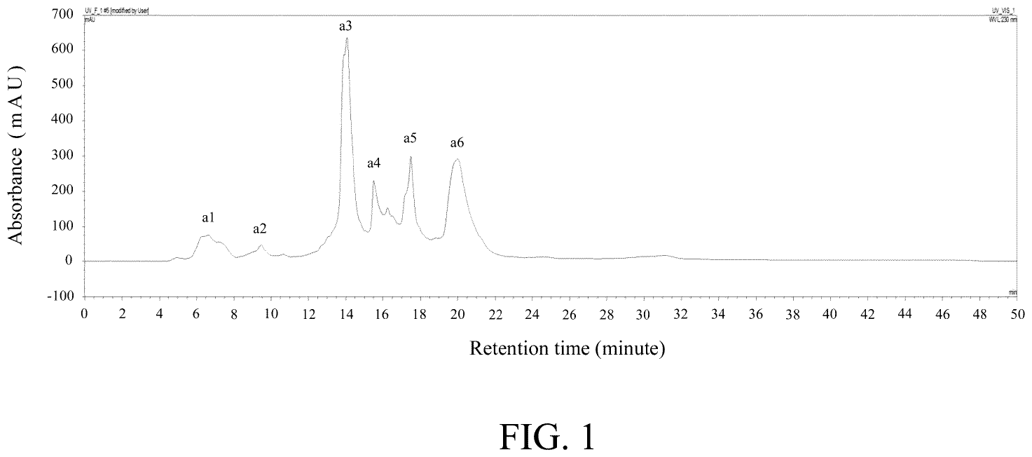

FIG. 1 shows the HPLC spectrum of the supercritical fluid-extracted product of Agaricus blazei Murrill. As shown in FIG. 1, there were six main peaks (i.e., peaks a1, a2, a3, a4, a5, and a6) during a 50-minute retention period, indicating that there were six major components in the supercritical fluid-extracted product of Agaricus blazei Murrill.

### Example 3. Evaluation for the Effect of Supercritical Fluid-Extracted Product of Agaricus blazei Murrill in Inhibiting the Growth of Cancer Cells

### Experimental Materials

1. Preparation of Test Solution of Ethanol-Extracted Product of Agaricus blazei Murrill

First, 30 g of the freeze-dried powder of Agaricus blazei Murrill obtained in Example 1 was mixed with 150 mL of 95% ethanol, followed by conducting ethanol extraction at 60° C. for 60 minutes. The resultant mixture was subsequently filtered through a filter paper with a porosity of 7 μm, so as to obtain a filtrate. The filtrate was then concentrated using a rotary evaporator (Manufacturer: Eyela, Model no.: N-1300) for 1 hour, so as to obtain the ethanol-extracted product of Agaricus blazei Murrill in pasty form.

Next, 100 mg of the ethanol-extracted product of Agaricus blazei Murrill was dissolved in 1 mL of 95% ethanol, followed by adding a suitable amount of DMEM, so as to obtain a test solution of the ethanol-extracted product of Agaricus blazei Murrill having a concentration of 400 μg/mL (abbreviated as a test solution E).

2. Preparation of Test Solutions of Water-Extracted Products of Agaricus blazei Murrill

First, 30 g of the freeze-dried powder of Agaricus blazei Murrill obtained in Example 1 was mixed with 180 ml of deionized water, followed by conducting water extraction at 80° C. for 60 minutes. The resultant mixture was subsequently filtered through a filter paper with a porosity of 7 μm, so as to obtain a filtrate. The filtrate was then concentrated using the rotary evaporator for 2 hours, followed by conducting a freeze-drying treatment for 48 hours, so as to obtain a dried powder of a water-extracted product of Agaricus blazei Murrill (abbreviated as a water-extracted product 1). Next, 100 mg of the dried powder of the water-extracted product 1 of Agaricus blazei Murrill was dissolved in 1 ml of the deionized water, followed by adding a suitable amount of DMEM, so as to obtain a test solution of the water-extracted product 1 of Agaricus blazei Murrill having a concentration of 400 μg/mL (abbreviated as a test solution W1).

In addition, a dried powder of a water-extracted product 2 of Agaricus blazei Murrill was prepared using procedures similar to those of the dried powder of the water-extracted product 1 of Agaricus blazei Murrill as described above, except that the water extraction was conducted at 4° C. for 60 minutes. Then, 100 mg of the dried powder of the water-extracted product 2 of Agaricus blazei Murrill was dissolved in 1 mL of the deionized water, followed by adding a suitable amount of the DMEM, so as to obtain a test solution of the water-extracted product 2 of Agaricus blazei Murrill having a concentration of 400 μg/mL (abbreviated as a test solution W2).

3. Preparation of Test Solution of Supercritical Fluid-Extracted Product of Agaricus blazei Murrill

Briefly, 100 mg of the supercritical fluid-extracted product of Agaricus blazei Murrill obtained in Example 1 was dissolved in 1 mL of 95% ethanol, followed by adding a suitable amount of the DMEM, so as to obtain a test solution of the supercritical fluid-extracted product of Agaricus blazei Murrill having a concentration of 400 μg/mL (abbreviated as a test solution S).

### Experimental Procedures

First, a respective one of the A549 cells, AGS cells, SW480 cells, and MDA-MB-231 cells prepared in section 1 of “General Experimental Materials” was divided into 5 groups (including one control group, one experimental group, and three comparative groups). Each group of the A549 cells, AGS cells, SW480 cells, and MDA-MB-231 cells was seeded in a respective well of a 96-well culture plate containing 100 μL of the corresponding medium as shown in Table 2 above, followed by cultivation in an incubator (37° C., 5% CO₂) for 18 hours.

Next, medium change was performed by adding the fresh corresponding medium as shown in Table 2 above into the respective well of the 96-well culture plate. Thereafter, the cell culture of each of the experimental groups and the comparative groups was treated with a suitable amount of the corresponding test solution as shown in Table 4 below, such that a final concentration of the treating agent in each group was 200 μg/mL. In addition, the cell culture of each of the control groups received no treatment.

TABLE 4

| | | Cell number | |
|---|---|---|---|
| Group | Cell line | (cells/well) | Treating agent |
| Control group 1 | A549 cells | 1.0 × 10³ | — |
| Experimental group 1 | | | Test solution S |
| Comparative group 1-1 | | | Test solution E |
| Comparative group 1-2 | | | Test solution W1 |
| Comparative group 1-3 | | | Test solution W2 |
| Control group 2 | AGS cells | 2.5 × 10³ | — |
| Experimental group 2 | | | Test solution S |
| Comparative group 2-1 | | | Test solution E |
| Comparative group 2-2 | | | Test solution W1 |
| Comparative group 2-3 | | | Test solution W2 |
| Control group 3 | SW480 | 6.0 × 10³ | — |
| Experimental group 3 | cells | | Test solution S |
| Comparative group 3-1 | | | Test solution E |
| Comparative group 3-2 | | | Test solution W1 |
| Comparative group 3-3 | | | Test solution W2 |
| Control group 4 | MDA-MB- | 4.0 × 10³ | — |
| Experimental group 4 | 231 cells | | Test solution S |
| Comparative group 4-1 | | | Test solution E |
| Comparative group 4-2 | | | Test solution W1 |
| Comparative group 4-3 | | | Test solution W2 |

After cultivation in an incubator (37° C., 5% CO₂) for 48 hours, the culture medium in each well was removed, and then the respective well was washed with phosphate-buffered saline (PBS) several times, followed by addition of 100 μL of water-soluble tetrazolium-1 (WST-1) reagent (Roche, Cat. no. 11644807001). After cultivation in an incubator (37° C., 5% CO₂) for 1 hour, the resultant mixture in each well was subjected to determination of absorbance at a wavelength of 450 nm (OD₄₅₀) using an ELISA reader (Manufacturer: Tecan, Model no.: Spark). In addition, a suitable amount of the corresponding medium as shown in Table 2 above was added into a respective well of a new 96-well culture plate, followed by subjecting the respective well to determination of absorbance at a wavelength of 450 nm (OD₄₅₀) using the ELISA reader, so as to obtain a background absorbance value.

The cell viability percentage (%) was calculated using the following Equation (1):

$$A = ( B - D ) / ( C - D ) \times 100 \qquad ( 1 )$$

where A=cell viability percentage (%)
   B=OD₄₅₀ value determined in each group
   C=OD₄₅₀ value determined in each control group
   D=background absorbance value determined for respective medium

The data thus obtained were analyzed according to the procedures as described in section 1 of “General Procedures.”

### Results

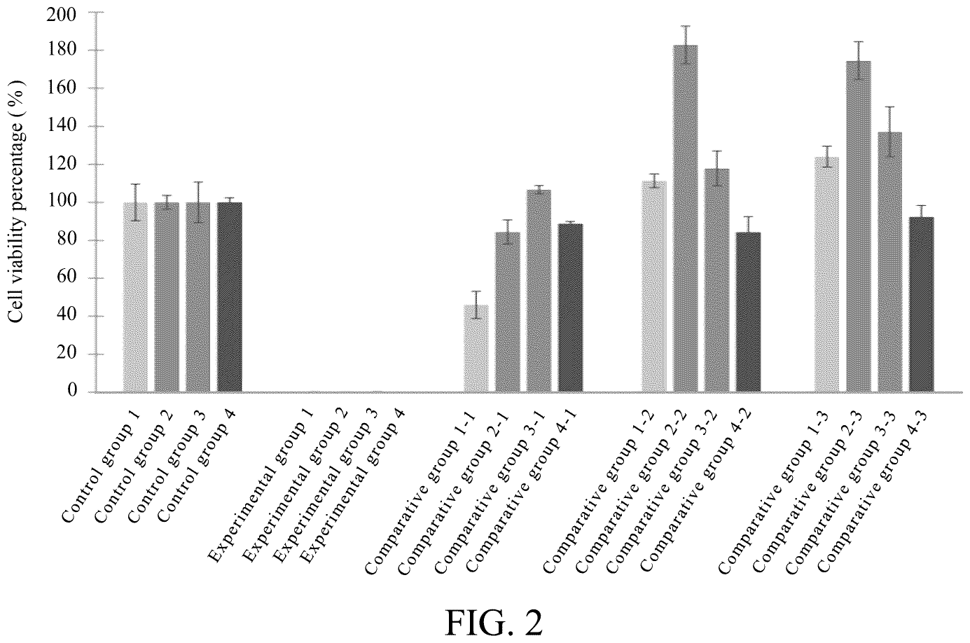

FIG. 2 shows the cell viability percentages determined in each group. As shown in FIG. 2, the cell viability percentage determined in each of the experimental groups 1 to 4 was significantly reduced to 0%. However, the cell viability percentage determined in the comparative groups 1-1 was slightly lower than that determined in the control group 1, the cell viability percentages determined in the comparative groups 1-2 and 1-3 were higher than that determined in the control group 1. Moreover, the cell viability percentage determined in the comparative group 2-1 showed no significant difference compared to the control group 2, and the cell viability percentage determined in the comparative group 3-1 showed no significant difference compared to the control group 3. The cell viability percentages determined in the comparative groups 2-2 and 2-3 were higher than that determined in the control group 2, and the cell viability percentages determined in the comparative groups 3-2 and 3-3 were higher than that determined in the control group 3. In addition, compared with the control group 4, the cell viability percentages determined in the comparative groups 4-1 to 4-3 showed no significant difference.

These results indicate that the supercritical fluid-extracted product of Agaricus blazei Murrill can exhibit excellent cytotoxicity against lung adenocarcinoma cells, gastric adenocarcinoma cells, colorectal cancer cells, and breast cancer cells, and hence is expected to be effective in inhibiting the growth of cancer cells.

### Example 4. Evaluation of the Effect of Mixture Solutions According to this Disclosure in Inhibiting the Growth of Cancer Cells In Vitro

### Experimental Materials

1. Preparation of Freeze-Dried Powder of Ethanol-Extracted Product of Curcuma longa

The ethanol-extracted product of Curcuma longa was prepared according to the method described in TW 1748565 B. Briefly, 30 g of a freeze-dried Curcuma longa rhizome powder was mixed with 150 ml of 95% ethanol, followed by conducting stirring extraction at 25° C. and 280 rpm for 30 minutes. The resultant mixture was subsequently filtered through a filter screen with 250 meshes, so as to obtain a filtrate. The filtrate was then concentrated using the rotary evaporator for 2 hours to remove the ethanol, followed by adding ethanol and mixing at 4° C., and then conducting a freeze-drying treatment for 48 hours, so as to obtain a freeze-dried powder of the ethanol-extracted product of Curcuma longa.

2. Preparation of Supercritical Fluid-Extracted Product of Ganoderma lucidum

A supercritical fluid-extracted product of Ganoderma lucidum was prepared using procedures similar to those of the supercritical fluid-extracted product of Agaricus blazei Murrill obtained in Example 1, except that the fruiting bodies of Agaricus blazei Murrill were replaced with the fruiting bodies of Ganoderma lucidum obtained from Zhongpu Township, Chiayi County, Taiwan, the amount of the freeze-dried powder of Ganoderma lucidum was adjusted to 4 kg, and the temperature of the supercritical fluid extraction (SFE) was adjusted to 40° C. The resultant supercritical fluid-extracted product of Ganoderma lucidum was in pasty form.

### 3. Preparation of Mixture Solutions

The supercritical fluid-extracted product of Agaricus blazei Murrill in pasty form obtained in Example 1, the freeze-dried powder of the ethanol-extracted product of Curcuma longa obtained in section 1 of “Experimental Materials” of Example 4, and the supercritical fluid-extracted product of Ganoderma lucidum in pasty form obtained in section 2 of “Experimental Materials” of Example 4 were mixed in specific combinations and weight ratios as shown in Table 5 below, so as to obtain 4 mixtures in pasty form (i.e., mixtures 1 to 4).

TABLE 5

| | | Weight |
|---|---|---|
| Mixture | Components | ratio |
| 1 | Supercritical fluid-extracted product of Agaricus | 1:1 |
| | blazei Murrill and supercritical fluid-extracted | |
| | product of Ganoderma lucidum | |
| 2 | Supercritical fluid-extracted product of Agaricus | 1:1 |
| | blazei Murrill and ethanol-extracted product of | |
| | Curcuma longa | |
| 3 | Supercritical fluid-extracted product of Agaricus | 1:1:1 |
| | blazei Murrill, ethanol-extracted product of | |
| | Curcuma longa, and supercritical fluid-extracted | |
| | product of Ganoderma lucidum | |
| 4 | Supercritical fluid-extracted product of Agaricus | 1:2:1 |
| | blazei Murrill, ethanol-extracted product of | |
| | Curcuma longa, and supercritical fluid-extracted | |
| | product of Ganoderma lucidum | |

Then, 100 mg of the respective one of the mixtures 1 to 4 was dissolved in 1 mL of 95% ethanol, followed by adding a suitable amount of DMEM, so as to obtain mixture solutions 1 to 4 with a respective concentration of 100 μg/mL, which were used in the following experiments.

### Experimental Procedures

### A. Comparison Between the Effects of Test Solution S and Mixture Solution 3 in Inhibiting the Growth of Cancer Cells

First, a respective one of the MIA Paca-2 cells, HeLa 229 cells, and PC-3 cells prepared in section 1 of “General Experimental Materials” was divided into 5 groups (including one control group and four experimental groups). Each group of the MIA Paca-2 cells, HeLa 229 cells, and PC-3 cells was seeded in a respective well of a 96-well culture plate containing 100 μL of the corresponding medium as shown in Table 2 above, followed by cultivation in an incubator (37° C., 5% CO₂) for 18 hours.

Next, medium change was performed by adding the fresh corresponding medium as shown in Table 2 above into the respective well of the 96-well culture plate. Thereafter, the cell culture of each of the experimental groups was treated with a suitable amount of the test solution S obtained in section 3 of “Experimental Materials” of Example 3 or the mixture solution 3, such that the treating agent had different final concentrations in each of the groups as shown in Table 6 below. In addition, the cell culture of each of the control groups 1 to 3 received no treatment.

TABLE 6

| | | Cell | | Final |
|---|---|---|---|---|
| | | number | Treating | concentration |
| Group | Cell line | (cells/well) | agent | (μg/mL) |
| Control group 1 | MIA Paca-2 | 5 x 10³ | — | — |
| Experimental | cells | | Test | 10 |
| group 1-1 | | | solution S | |
| Experimental | | | | 100 |
| group 1-2 | | | | |
| Experimental | | | Mixture | 10 |
| group 1-3 | | | solution 3 | |
| Experimental | | | | 100 |
| group 1-4 | | | | |
| Control group 2 | HeLa 229 | 5 × 10³ | — | — |
| Experimental | cells | | Test | 10 |
| group 2-1 | | | solution S | |
| Experimental | | | | 100 |
| group 2-2 | | | | |
| Experimental group | | | Mixture | 10 |
| 2-3 | | | solution 3 | |
| Experimental | | | | 100 |
| group 2-4 | | | | |
| Control group 3 | PC-3 cells | 5 × 10³ | — | — |
| Experimental | | | Test | 10 |
| group 3-1 | | | solution S | |
| Experimental | | | | 100 |
| group 3-2 | | | | |
| Experimental | | | Mixture | 10 |
| group 3-3 | | | solution 3 | |
| Experimental | | | | 100 |
| group 3-4 | | | | |

After cultivation in an incubator (37° C., 5% CO₂) for 48 hours, the cell culture of each group was subjected to sulforhodamine B (SRB) assay using an In Vitro Toxicology Assay Kit (Sigma, Cat. no. TOX6-1KT). Briefly, the cell culture of each group was subjected to a fixation treatment with 50 μL of trichloroacetic acid (TCA) solution at 4° C. for 1 hour. The liquid in each well was then removed, and the respective well was washed with sterilized water, followed by addition of 50 μL of 0.4% SRB reagent at room temperature for 30 minutes. Subsequently, the liquid in each well was removed, and the respective well was washed with 1% acetic acid, followed by addition of 100 μL of 10 mM Tris-base and placement on a digital orbital shaker (Manufacturer: DLAB Scientific Inc., Model no.: SK-0180-S) for 30 minutes to mix well. The resultant mixture in each well was subsequently subjected to determination of absorbance at a wavelength of 565 nm (OD₅₆₅) using the ELISA reader. In addition, a suitable amount of the corresponding medium as shown in Table 2 above was added into a respective well of a new 96-well culture plate, followed by subjecting the respective well to determination of absorbance at a wavelength of 565 nm (OD₅₆₅) using the ELISA reader, so as to obtain a background absorbance value.

The cell viability percentage (%) was calculated using the following Equation (2):

$$E = ( F - H ) / ( G - H ) \times 100 \qquad ( 2 )$$

where E=cell viability percentage (%)
   F=OD₅₆₅ value determined in each group
   G=OD₅₆₅ value determined in each control group
   H=background absorbance value determined for respective medium

The data thus obtained were analyzed according to the procedures as described in section 1 of “General Procedures.”

### B. Comparison Between the Effects of Mixture Solutions 1 to 3 in Inhibiting the Growth of Cancer Cells

First, a respective one of the A549 cells, AGS cells, HepG2 cells, SW480 cells, and MDA-MB-231 cells prepared in section 1 of “General Experimental Materials” was divided into 4 groups (including one control group and three experimental groups). Each group of the A549 cells, AGS cells, HepG2 cells, SW480 cells, and MDA-MB-231 cells was seeded in a respective well of a 96-well culture plate containing 100 μL of the corresponding medium as shown in Table 2 above, followed by cultivation in an incubator (37° C., 5% CO₂) for 18 hours.

Next, medium change was performed by adding the fresh corresponding medium as shown in Table 2 above into the 96-well culture plate. Thereafter, the cell culture of each of the experimental groups was treated with a suitable amount of the corresponding mixture solutions 1 to 3 as shown in Table 7 below, such that a final concentration of the treating agent in each group was 200 μg/mL. In addition, the cell culture of each of the control groups 1 to 5 received no treatment.

TABLE 7

| | | Cell number | |
|---|---|---|---|
| Group | Cell line | (cells/well) | Treating agent |
| Control group 1 | A549 cells | 1.0 × 10³ | |
| Experimental | | | Mixture solution 1 |
| group 1-1 | | | |
| Experimental | | | Mixture solution 2 |
| group 1-2 | | | |
| Experimental | | | Mixture solution 3 |
| group 1-3 | | | |
| Control group 2 | AGS cells | 2.5 × 10³ | |
| Experimental | | | Mixture solution 1 |
| group 2-1 | | | |
| Experimental | | | Mixture solution 2 |
| group 2-2 | | | |
| Experimental | | | Mixture solution 3 |
| group 2-3 | | | |
| Control group 3 | HepG2 | 1.3 × 10⁴ | |
| Experimental | cells | | Mixture solution 1 |
| group 3-1 | | | |
| Experimental | | | Mixture solution 2 |
| group 3-2 | | | |
| Experimental | | | Mixture solution 3 |
| group 3-3 | | | |
| Control group 4 | SW480 | 6.0 × 10³ | |
| Experimental | cells | | Mixture solution 1 |
| group 4-1 | | | |
| Experimental | | | Mixture solution 2 |
| group 4-2 | | | |
| Experimental | | | Mixture solution 3 |
| group 4-3 | | | |
| Control group 5 | MDA-MB- | 4.0 × 10³ | |
| Experimental | 231 cells | | Mixture solution 1 |
| group 5-1 | | | |
| Experimental | | | Mixture solution 2 |
| group 5-2 | | | |
| Experimental | | | Mixture solution 3 |
| group 5-3 | | | |

After cultivation in an incubator (37° C., 5% CO₂) for 48 hours, the cell culture of the respective group was subjected to determination of cell viability percentage according to the procedures described in “Experimental Procedures” of Example 3.

### Results

### A. Comparison Between the Effects of Test Solution S and Mixture Solution 3 in Inhibiting the Growth of Cancer Cells

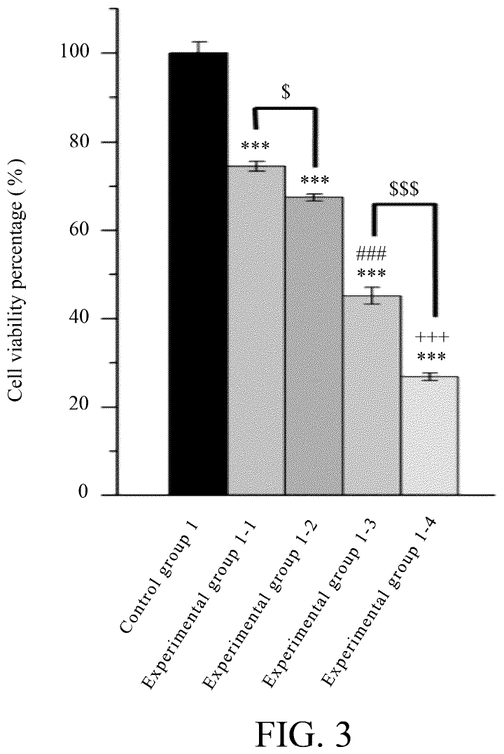

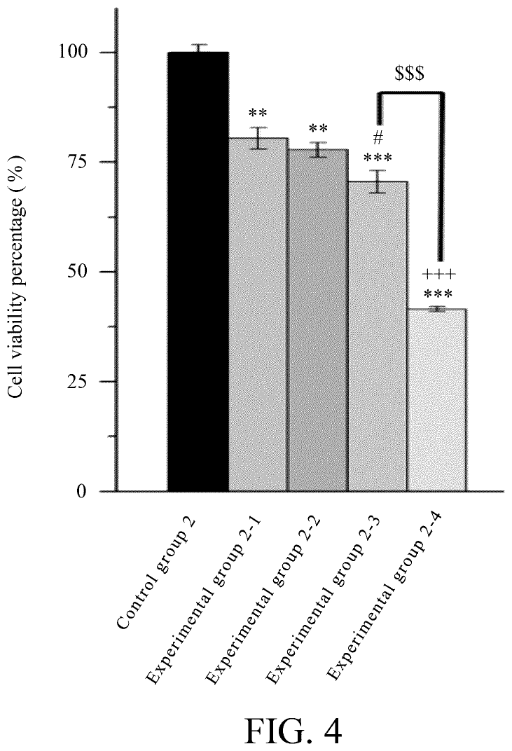

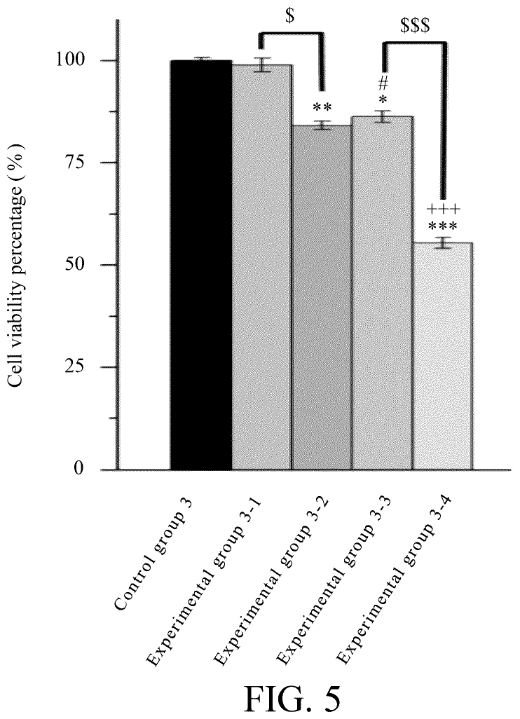

FIGS. 3 to 5 show the cell viability percentages determined in each group. As shown in FIG. 3, compared with the control group 1, the cell viability percentage determined in each of the experimental groups 1-1 to 1-4 was reduced. In particular, the cell viability percentage determined in each of the experimental groups 1-3 and 1-4 was significantly lower than that determined in the control group 1. In addition, as shown in FIGS. 4 and 5, similar results were observed with respect to the experimental groups 2-3 and 2-4, and 3-3 and 3-4 compared to the respective control groups 2 and 3.

These results indicate that the supercritical fluid-extracted product of Agaricus blazei Murrill, either alone or in combination with the ethanol-extracted product of Curcuma longa and the supercritical fluid-extracted product of Ganoderma lucidum, are capable of effectively inhibiting the growth of the pancreatic cancer cells, cervical cancer cells, and prostate cancer cells. Moreover, a combination of the supercritical fluid-extracted product of Agaricus blazei Murrill, the ethanol-extracted product of Curcuma longa and the supercritical fluid-extracted product of Ganoderma lucidum can exhibit dose-related synergistic anti-cancer effect.

### B. Comparison Between the Effects of Mixture Solutions 1 to 3 in Inhibiting the Growth of Cancer Cells

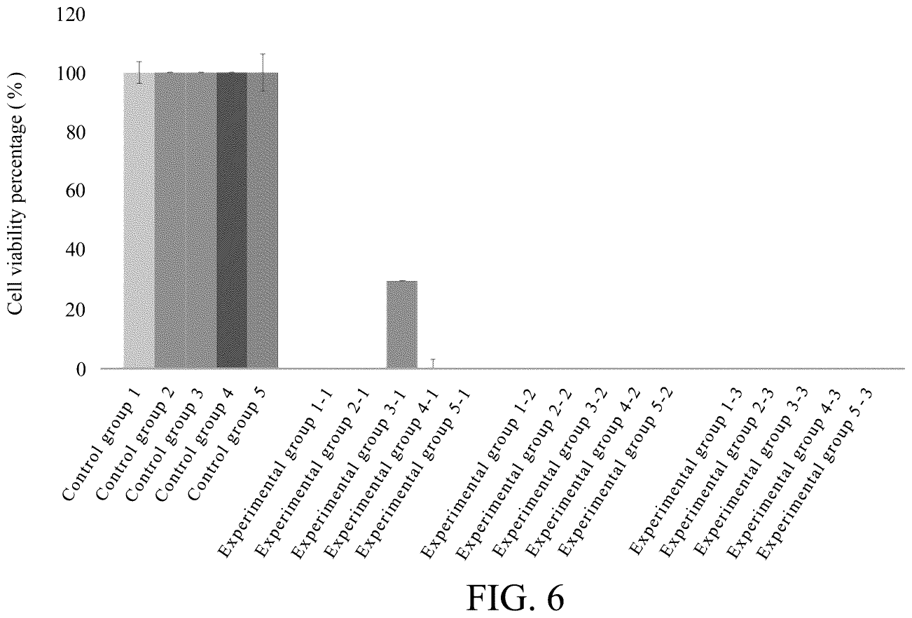

FIG. 6 shows the cell viability percentages determined in each group. As shown in FIG. 6, compared with the control group 1, the cell viability percentage determined in each of the experimental groups 1-1 to 1-3 was significantly reduced. Besides, similar results were observed with respect to the experimental groups 2-1 to 2-3, 3-1 to 3-3, 4-1 to 4-3, and 5-1 to 5-3 compared to the respective control groups 2 to 5.

These results indicate that the supercritical fluid-extracted product of Agaricus blazei Murrill, either in combination with the ethanol-extracted product of Curcuma longa or the supercritical fluid-extracted product of Ganoderma lucidum, or in combination with both, are capable of effectively inhibiting the growth of the lung adenocarcinoma cells, gastric adenocarcinoma cells, liver cancer cells, colorectal cancer cells, and breast cancer cells.

### Example 5. Evaluation of the Effect of Mixture Solutions According to this Disclosure in Inhibiting the Growth of Cancer Cells In Vivo

### Experimental Materials

### 1. Experimental Mice

Male Balb/cAnN. Cg-Foxnl^{nu}/CrlNarl nude mice (5 weeks old, with a body weight of approximately 19.61±1.36 g) used in the following experiments were purchased from National Laboratory Animal Center, R.O.C. All the experimental mice were housed in an animal room with an independent air conditioning system under the following laboratory conditions: an alternating 12-hour light and 12-hour dark cycle, a temperature maintained at 22±2° C., and a relative humidity maintained at 55±15%. The mice were provided with water and fed ad libitum. After 1 week of feeding, the mice were subjected to the experiments described below. All experimental procedures involving the experimental mice were in compliance with the legal provision of the Animal Protection Act of Taiwan, and were carried out according to the guidelines of the Animal Care Committee of the Council of Agriculture, Taiwan.

### 2. Preparation of A549 Cell Solution

The A549 cells prepared in section 1 of “General Experimental Materials” were centrifuged at 5000 rpm and 25° C. for 5 minutes, and then the resultant cell pellet were collected. Subsequently, 1 mL of the fresh corresponding medium as shown in Table 2 above was added to suspend the cell pellet, so as to obtain a suspension. Then, 10 μL of the suspension was diluted with 990 μL of the PBS, followed by adjusting to the desired cell concentration which was determined using a plate counting medium, thereby obtaining an A549 cell solution having 1×10⁷ cells.

### 3. Preparation of MIA Paca-2 Cell Solution

The MIA Paca-2 cells prepared in section 1 of “General Experimental Materials” were centrifuged at 5000 rpm and 25° C. for 5 minutes, and then the resultant cell pellet were collected. Subsequently, 1 ml of the fresh corresponding medium as shown in Table 2 above was added to suspend the cell pellet, so as to obtain a suspension. Then, 10 μL of the suspension was diluted with 990 μL of the PBS, followed by adjusting to the desired cell concentration which was determined using a plate counting medium, thereby obtaining an MIA Paca-2 cell solution having 1×10⁷ cells.

### 4. Preparation of Mixture Solution 3-1

A suitable amount of the mixture 3 in pasty form obtained in section 3 of “Experimental Materials” of Example 4 was dissolved in a suitable amount of DMSO (J. T. Baker, Cat. no. 9224-01), followed by adding a suitable amount of sterilized distilled water, so as to obtain the mixture solution 3-1.

### 5. Preparation of Mixture Solution 3-2

The encapsulation of the mixture 3 in pasty form obtained in section 3 of “Experimental Materials” of Example 4 into a liposome was performed by partially referring the method described in Lombardo D. and Kiselev M. A. (2022), Pharmaceutics., doi: 10.3390/pharmaceutics14030543, so as to obtain a mixture 3 in powder form. The suitable amount of the mixture 3 in powder form was dissolved in a suitable amount of the sterilized distilled water, so as to obtain the mixture solution 3-2.

### Experimental Procedures

### A. Evaluation of Efficacy Against Lung Adenocarcinoma Cells

Each nude mouse was subcutaneously injected with the A549 cell solution obtained in section 2 of “Experimental Materials” of Example 5 near the dorsal side of the right hind limb. On the 14th day post-injection, these nude mice with tumors ranging from 5 mm to 6 mm in diameter were randomly divided into 6 groups (n=8 mice in each group), including a control group and 5 experimental groups (i.e., experimental groups 1 to 5). Thereafter, the nude mice in each of the experimental groups 1 to 5 were fed, via oral gavage, with the appropriate amount of the test solution S obtained in section 3 of “Experimental Materials” of Example 3 or the mixture solutions 1 to 4 prepared in section 3 of “Experimental Materials” of Example 4, so that each nude mouse in the experimental groups 1 to 5 received a dose of the treating agent per day as shown in Table 8 below. Each nude mouse was fed once daily for a total period of 35 days. In addition, each nude mouse in the control group received no treatment.

TABLE 8

| | | Dose |
|---|---|---|
| Group | Treating agent | (mg/kg/day) |
| Control group | — | — |
| Experimental | Supercritical fluid-extracted product of | 100 |
| group 1 | Agaricus blazei Murrill | |
| Experimental | Mixture 1 | 200 |
| group 2 | | |
| Experimental | Mixture 2 | 200 |
| group 3 | | |
| Experimental | Mixture 3 | 200 |
| group 4 | | |
| Experimental | Mixture 4 | 200 |
| group 5 | | |

Prior to the feeding of the treating agent (i.e., on the 0^{th} day) and after the feeding of the same (i.e., on the 7^{th}, 14^{th}, 21^{rd}, 28^{th}, and 35^{th} days), each nude mouse was subjected to determination of tumor volume (mm³) using a tumor scanner (Manufacturer: Peira, Model no.: TM900).

The data thus obtained were analyzed according to the procedures as described in section 1 of “General Procedures.”

### B. Evaluation of Efficacy Against Pancreatic Cancer Cells

Each nude mouse was subcutaneously injected with the MIA Paca-2 cell solution obtained in section 3 of “Experimental Materials” of Example 5 near the dorsal side of the right hind limb. On the 14^{th} day post-injection, these nude mice with tumors ranging from 5 mm to 6 mm in diameter were randomly divided into 6 groups (n=8 mice in each group), including a control group and 5 experimental groups (i.e., experimental groups 1 to 5).

Thereafter, the nude mice in each of the experimental groups 1 to 5 were fed, via oral gavage, with the appropriate amount of the mixture solution 3-1 obtained in section 4 of “Experimental Materials” of Example 5 or the mixture solution 3-2 obtained in section 5 of “Experimental Materials” of Example 5, so that each nude mouse in the experimental groups 1 to 5 received a dose of the treating agent per day as shown in Table 9 below. Each nude mouse was fed once daily for a total period of 35 days. In addition, each mouse in the control group received no treatment.

TABLE 9

| | | Dose |
|---|---|---|
| Group | Treating agent | (mg/kg/day) |
| Control group | — | — |
| Experimental | Mixture 3 in pasty form | 200 |
| group 1 | | |
| Experimental | | 400 |
| group 2 | | |
| Experimental | | 800 |
| group 3 | | |
| Experimental | Mixture 3 in powder form | 200 |
| group 4 | | |
| Experimental | | 800 |
| group 5 | | |

Prior to the feeding of the treating agent (i.e., on the 0^{th} day) and after the feeding of the same (i.e., on the 7^{th}, 14^{th}, 21^{rd}, 28^{th}, and 35^{th} days), each nude mouse was subjected to determination of tumor volume (mm³) using the tumor scanner.

The data thus obtained were analyzed according to the procedures as described in section 1 of “General Procedures.”

### Results

### A. Evaluation of Efficacy Against Lung Adenocarcinoma Cells

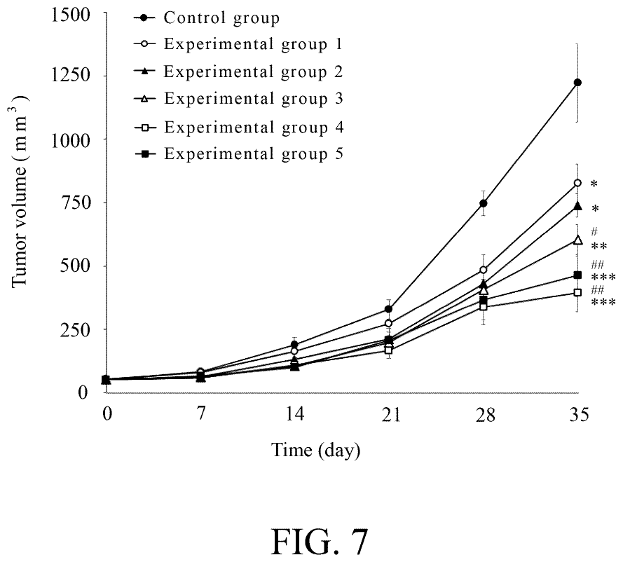

FIG. 7 shows the changes in A549 tumor volume over time determined in each group. As shown in FIG. 7, the A549 tumor volume determined in the control group gradually increased over time, whereas those determined in the experimental groups 1 to 5 showed slow increases over time. In particular, the A549 tumor volume determined in the experimental groups 4 and 5 showed the slowest rates of increase.

These results demonstrate that the supercritical fluid-extracted product of Agaricus blazei Murrill, either alone or in combination with the ethanol-extracted product of Curcuma longa or the supercritical fluid-extracted product of Ganoderma lucidum, or in combination with both, are capable of effectively inhibiting the growth of the lung adenocarcinoma cells in vivo.

### B. Evaluation of Efficacy Against Pancreatic Cancer Cells

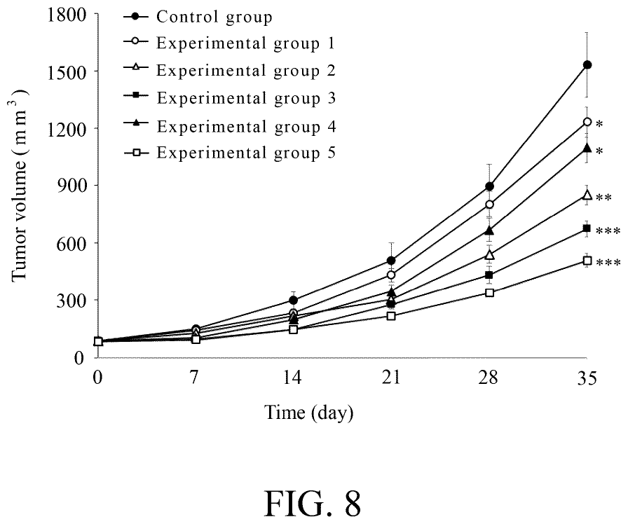

FIG. 8 shows the changes in MIA-Paca-2 tumor volume over time determined in each group. As shown in FIG. 8, the MIA-Paca-2 tumor volume determined in the control group gradually increased over time, whereas those determined in the experimental groups 1 to 5 showed slow increases over time.

These results indicate that the combination of the supercritical fluid-extracted product of Agaricus blazei Murrill with the ethanol-extracted product of Curcuma longa and the supercritical fluid-extracted product of Ganoderma lucidum, whether in pasty or powder form, is capable of effectively inhibiting the growth of the pancreatic cancer cells in vivo.

Summarizing the above test results, it is clear that the supercritical fluid-extracted product of Agaricus blazei Murrill can effectively inhibit the growth of cancer cells, including the lung adenocarcinoma cells, the gastric adenocarcinoma cells, the colorectal cancer cells, the breast cancer cells, the liver cancer cells, the pancreatic cancer cells, the prostate cancer cells, and the cervical cancer cells. Moreover, the supercritical fluid-extracted product of Agaricus blazei Murrill, when used in combination with the ethanol-extracted product of Curcuma longa or the supercritical fluid-extracted product of Ganoderma lucidum, or in combination with both, can exhibit synergistic anti-cancer effect.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for inhibiting the growth of cancer cells, comprising administering to a subject in need thereof a pharmaceutical composition containing a supercritical fluid-extracted product of Agaricus blazei Murrill.

2. The method as claimed in claim 1, wherein the cancer cells are selected from the group consisting of lung adenocarcinoma cells, gastric adenocarcinoma cells, colorectal cancer cells, breast cancer cells, liver cancer cells, pancreatic cancer cells, prostate cancer cells, cervical cancer cells, and combinations thereof.

3. The method as claimed in claim 1, wherein the pharmaceutical composition further contains an extracted product selected from the group consisting of an ethanol-extracted product of Curcuma longa, a supercritical fluid-extracted product of Ganoderma lucidum, and a combination thereof.

4. The method as claimed in claim 3, wherein the pharmaceutical composition contains the supercritical fluid-extracted product of Agaricus blazei Murrill, the ethanol-extracted product of Curcuma longa, and the supercritical fluid-extracted product of Ganoderma lucidum.

5. The method as claimed in claim 1, wherein the pharmaceutical composition is in a dosage form selected from the group consisting of a parenteral dosage form, an oral dosage form, and a topical dosage form.
